(19) United States
(12) Patent Application Publication
(10) Pub. No.: US 2007/0238142 A1
(43) Pub. Date: Oct. 11, 2007

(54) Small molecule mediated biosensing using carbon nanotubes in homogeneous format

(76) Inventors: Salah Boussaad, Wilmington, DE (US); Bruce A. Diner, Chadds Ford, PA (US); Janine Fan, Hockessin, DE (US); Vsevolod Rostovtsev, Swarthmore, PA (US)

Correspondence Address: E I DU PONT DE NEMOURS AND COMPANY;LEGAL PATENT RECORDS CENTER, BARLEY MILL PLAZA 25/1128, 4417 LANCASTER PIKE, WILMINGTON, DE, 19805, US

(21) Appl. No.: 11/241,558

(22) Filed: Sep. 30, 2005

Related U.S. Application Data
(60) Provisional application No. 60/615,309, filed on Sep. 30, 2004.

(51) Int. Cl. C12Q 1/54; C12Q 1/26; C12M 3/00

(52) U.S. Cl. 435/14; 435/25; 435/287.1; 977/902; 205/777.5

(57) ABSTRACT

Nanosensors for detecting target analytes and methods of detecting analytes have been developed in which a small molecule effector concentration is altered thereby causing changes in carbon nanotube conductance. The nanosensor operates in a homogeneous format, not requiring the immobilization of the target analyte for detection.

### FIELD OF INVENTION

This invention relates to the field of nanotechnology. Specifically the invention describes a nanosensor for the detection of analytes in which the concentration of a small molecule effector is altered thereby causing changes in the conductance of a carbon nanotube.

### BACKGROUND OF THE INVENTION

There is an increasing need for rapid, small scale and highly sensitive detection of biological molecules in medical, bioterrorism, food safety, and research applications. Nanostructures such as silicon nanowires and carbon nanotubes display physical and electronic properties amenable to use in miniature devices. Carbon nanotubes (CNTs) are rolled up graphene sheets having a diameter on the nanometer scale and typical lengths of up to several micrometers. CNTs can behave as semiconductors or metals depending on their chirality. Additionally, dissimilar carbon nanotubes may contact each other allowing the formation of a conductive path with interesting electrical, magnetic, nonlinear optical, thermal and mechanical properties.

It is known that single-walled carbon nanotubes are sensitive to their chemical environment, specifically that exposure to air or oxygen alters their electrical properties (Collins et al. (2000) Science 287:1801). Additionally, exposure of CNTs to gas molecules such as NO₂ or NH₃ alters their electrical conductance (Kong et al. (2000) Science 287:622). Thus chemical gas sensors can be designed on the basis of the electrical properties of carbon nanotubes such as described in DE10118200.

Sanjay and Kramer ((1996) Nature Biotech. 14:303) describe the detection of DNA in solution using molecular beacons. These are stem-loop structures that contain a fluorescence emitter and quencher, one on each strand at the base of the stem, that open in the presence of a DNA single strand or RNA, complementary to the loop region, producing an increase in the fluorescence yield of the emission. Used for real-time PCR, these structures produce a dequenching of one fluorescence emitter for every complementary nucleic acid strand hybridized.

In WO 02/48701 articles are described that use nanowires, including CNTs, to detect different types of analytes including biological analytes. The nanowire may be modified by attaching an agent that is designed to bind an analyte, the binding to the nanowire or to a coating on the nanowire then causes a detectable change in conductance. In this detection system the interaction between the binding agent and the analyte to be detected alters the electrical conductance of the nanowire. This requirement in turn limits the functional location of the binding agent with respect to the nanowire in that they must be in close proximity, 5 nanometers or less.

There is a need for a nanoscale detection system that has the ability to indirectly detect an analyte in a solution-based format that can provide a signal the concentration of which exceeds by one or more orders of magnitude the concentration of the analyte. Applicants have solved this problem by developing a carbon nanotube based nanosensor that responds to a target analyte by producing or consuming an effector in solution that causes a change in the electrical conductance of the CNT. The concentration of effector produced or consumed far exceeds that of the analyte. The nanosensor operates in a homogenous format, not requiring the immobilization of the analyte for detection.

### SUMMARY OF THE INVENTION

The present invention provides a nanosensor for the detection of an analyte. The nanosensor comprises an electrically conducting path of semiconducting single-walled carbon nanotubes having a baseline conductance, in contact with a solution including a small molecule effector. Alterations in the concentration of the effector molecule alter the conductance of the CNTs with respect to the baseline conductance, thereby producing a quantifiable signal that can be correlated to the presence of the analyte. In a first embodiment the concentration of the effector is altered in the presence of the analyte by a reporter molecule in solution that interacts with the analyte.

In a second embodiment, contact of a reporter substrate with a catalytic analyte in solution causes a change in effector concentration. In a third embodiment the analyte itself causes a change in the concentration of the effector. In a fourth embodiment the presence of the analyte turns on a catalytic reporter that is modified with an activity switch such that it is turned off in the absence of the analyte. Upon activation the reporter is then able to catalyze a reaction between a reporter substrate and the effector. The invention also provides methods for detecting an analyte through the detection of an effector whose concentration is altered in the presence of a reporter molecule, a reporter substrate, or directly by an analyte. In the first, second and fourth embodiments, the change in the effector concentration is amplified such that its change in concentration greatly exceeds that of the analyte. Because the changes in effector concentration occur in solution, the reaction coupled to the presence of the analyte and detecting its presence occurs at a distance from the CNTs.

Accordingly the invention provides a nanosensor for detecting the presence of an analyte comprising:
a) at least two electrodes connected by an electrically conducting path comprised of one or more carbon nanotubes wherein at least one of said carbon nanotubes is semiconducting, and wherein the carbon nanotube is in contact with an effector; and
b) at least one reporter molecule having an analyte as a reporter substrate.

In an alternate embodiment the invention provides a nanosensor for detecting the presence of a catalytic analyte comprising:
a) at least two electrodes connected by an electrically conducting path comprised of one or more carbon nanotubes wherein at least one of said carbon nanotubes is semiconducting, and wherein the carbon nanotube is in contact with an effector; and
b) a reporter substrate that is a substrate of an catalytic analyte.

In a similar embodiment the invention provides a nanosensor for detecting the presence of an analyte comprising:
a) at least two electrodes connected by an electrically conducting path comprised of one or more carbon nanotubes wherein at least one of said carbon nanotubes is semiconducting, wherein the carbon nanotube is in contact with an effector; and
b) an effector responsive to the presence of an analyte.

In another embodiment the invention provides a nanosensor for detecting the presence of an analyte comprising:
a) at least two electrodes connected by an electrically conducting path comprised of one or more carbon nanotubes wherein at least one of said carbon nanotubes is semiconducting, wherein the carbon nanotube is in contact with an effector;
b) a reporter molecule comprising an activity switch comprising an analyte receptor linked to a reporter inhibitor; and
c) a reporter substrate that is a substrate of the reporter molecule.

Methods of the invention include:

A method for detecting an analyte comprising:
a) providing a nanosensor comprising:
   i) at least two electrodes connected by an electrically conducting path comprised of one or more carbon nanotubes wherein at least one of said carbon nanotubes is semiconducting, and wherein the carbon nanotube is in contact with an effector and has a baseline conductance; and
   ii) a reporter molecule having an analyte as a substrate;
b) providing a sample suspected of containing an analyte;
c) contacting the sample of (b) with the reporter molecule of (a) wherein the concentration of the effector molecule is altered resulting in a change in the conductance of the carbon nanotube with respect to the baseline conductance; and
d) measuring the change in conductance of the carbon nanotube with respect to the baseline conductance whereby the presence of the analyte is detected;

as well as;

A method for detecting a catalytic analyte comprising:
a) providing a nanosensor comprising:
   i) at least two electrodes connected by an electrically conducting path comprised of one or more carbon nanotubes wherein at least one of said carbon nanotubes is semiconducting, and wherein the carbon nanotube is in contact with an effector and has a baseline conductance; and
   ii) a reporter substrate that is a substrate of a catalytic analyte;
b) providing a sample suspected of containing a catalytic analyte;
c) contacting the sample of (b) with the reporter substrate of (a) wherein the concentration of the effector molecule is altered resulting in a change in the conductance of the carbon nanotube with respect to the baseline conductance; and
d) measuring the change in conductance of the carbon nanotube with respect to the baseline conductance whereby the presence of the catalytic analyte is detected;

as well as;

A method for detecting an analyte comprising:
a) providing a nanosensor comprising:
   i) at least two electrodes connected by an electrically conducting path comprised of one or more carbon nanotubes wherein at least one of said carbon nanotubes is semiconducting, and wherein the carbon nanotube is in contact with an effector and has a baseline conductance; and
   ii) an effector responsive to the presence of an analyte;
b) providing a sample suspected of containing an analyte;
c) contacting the sample of (b) with the effector of (a) wherein the concentration of the effector molecule is altered resulting in a change in the conductance of the carbon nanotube with respect to the baseline conductance; and
d) measuring the change in conductance of the carbon nanotube with respect to the baseline conductance whereby the presence of the analyte is detected;

as well as;

A method for detecting an analyte comprising:
a) providing a nanosensor comprising:
   i) at least two electrodes connected by an electrically conducting path comprised of one or more carbon nanotubes wherein at least one of said carbon nanotubes is semiconducting, and wherein the carbon nanotube is in contact with an effector and has a baseline conductance; and
   ii) a reporter molecule having an activity switch comprising an analyte receptor linked to a reporter inhibitor;
b) providing a sample suspected of containing an analyte which binds to the analyte receptor of the activity switch wherein the reporter molecule becomes active;
c) contacting the sample of (b) with the reporter molecule of (a) wherein the concentration of the effector molecule is altered resulting in a change in the conductance of the carbon nanotube with respect to the baseline conductance; and
d) measuring the change in conductance of the carbon nanotube with respect to the baseline conductance whereby the presence of the analyte is detected.

### BRIEF DESCRIPTION OF THE FIGURES

The invention can be more fully understood from the following detailed description and the accompanying sequence descriptions, which form a part of this application.

FIG. 1 is a diagram of a nanosensor embodiment with a reporter in solution.

FIG. 2 is a diagram of a nanosensor embodiment with a reporter substrate in solution.

FIG. 3 is a diagram of a nanosensor embodiment with an analyte directly changing the effector concentration.

FIG. 4 is a diagram of a nanosensor embodiment with a modified catalytic reporter that is turned on by the binding of a nucleic acid analyte.

FIG. 5 shows plots of the source-drain current of a CNT conducting path vs gate voltage recorded in air and nitrogen. The bias voltage is 1 V.

FIG. 6 shows plots of the source-drain current of a CNT conducting path vs liquid gate voltages recorded in buffer equilibrated with nitrogen (A) or with air (B) atmospheres as function of time following reduction of the CNTs. The bias voltage is 0.05 V.

FIG. 7 shows plots of the source-drain current of a CNT conducting path vs. liquid gate voltage before and after a pH jump. The bias voltage is 0.05 V.

### DETAILED DESCRIPTION

The present invention provides nanosensors for the detection of analytes. Typically analytes for the purposes of the invention are biomolecules. In the present invention CNTs are used to detect the presence of an analyte by responding to a change in the concentration of a small molecule effector. The conductance of the CNTs of the nanosensor will evolve from a baseline conductance with changes in concentration of the effector molecule. The main elements of the nanosensor of the invention are:

An electrically conducting path between at least two electrodes comprised of at least one semiconducting CNT where the CNT has a baseline conductance;

An effector molecule in association with the CNT where alterations in the concentration of the effector will alter the conductance of the CNT; and

Optionally, a reporter, typically a catalyst, that changes the concentration of the effector. An analyte may itself act as the reporter.

In contrast to previous methods the detection does not involve direct binding of the target biomolecule on or in close proximity to the CNT. The concentration of the effector is changed by a reporter molecule or a reporter substrate which interacts with an analyte. Alternatively, an analyte activates an inhibited reporter molecule in the presence of a reporter substrate. The interaction, which alters the concentration of the effector, changes the conductance of at least one semiconducting CNT in contact with a solution containing the effector molecules. Alternatively, an analyte may directly change the concentration of the effector. Advantages of this detection system are that 1) the target analyte alone or in a complex with a reporter molecule does not need to be attached to or be in close proximity to the CNT and 2) the effect on the effector concentration, caused by the presence of the analyte, is magnified relative to the concentration of the analyte.

The present invention also provides methods for detecting an analyte indirectly by introducing a reporter, a reporter substrate, or an inhibited reporter and reporter substrate that interacts with a target analyte causing a change in an effector concentration, and then measuring the change in conductance of at least one CNT in a conductive path that is in contact with a solution containing the effector.

Highly sensitive nanoscale detection of biomolecules has utility in bioterrorism, biomedical, environmental, food safety, research, and other applications. Use of the present system wherein detection by the CNT is of a change of effector concentration in solution increases the diversity of biomolecules that may be assayed and the sensitivity of detection. Samples may be screened to detect a target biomolecule that would provide information regarding a bioterrorism agent, a disease agent, a genetic disorder, an environmental contaminant, a food pathogen, a desired product, and other such components.

The following definitions and abbreviations are to be used for the interpretation of the claims and the specification.

“CNT” means carbon nanotube.

The term “nanotube” refers to a single-walled hollow cylinder having a diameter on the nanometer scale and a length of several micrometers, where the ratio of the length to the diameter, i.e., the aspect ratio, is at least 5. In general, the aspect ratio is between 100 and 100000.

By “carbon-based nanotube” or “carbon nanotube” herein is meant a single-walled hollow cylinder composed primarily of carbon atoms.

The term “baseline conductance” refers to the conductance of a carbon nanotube comprised within a nanosensor of the invention, measured prior to the addition of the sample or at the earliest time following the addition of a solution potentially containing the analyte for detection. The baseline conductance provides a measurement that can be compared to the conductance measurement made when the analyte is being detected.

The term “activity switch” refers to an aspect of a reporter molecule that allows permits the presence of an analyte to activate the reporter. Typically the activity switch comprises two elements, an “analyte receptor” and an “inhibitor” or “reporter inhibitor”. The analyte receptor generally will incorporate the inhibitor. In the absence of analyte the activity switch function to inhibit the reporter in that the inhibitor blocks the active site of the reporter. In the presence of an analyte the activity switch is modified such that the inhibitor is removed from the active site and the reporter is activated. “Analyte receptors” are any element that can be fixed to the reporter and that will bind the analyte. Additionally the analyte receptor must be able to comprise the inhibitor. Typical analyte receptors are biomolecules such as oligonucleotides, peptides, proteins, and peptide nucleic acids. Where the reporter is an enzyme, inhibitors will be enzyme inhibitors.

The term “homogeneous” as used in conjunction with the nanosenor and methods of the invention refers to a sensor or method that makes use of reagents in solution. The “homogeneous catalysis” refers to catalysis by a free catalytic moiety in a solution.

The term “analyte” or target analyte” means the substance that is the object of detection by the nanosensor. Analytes may be a variety of materials and substances but are typically biomolecules and the products of biological reactions and events. A “catalytic analyte” for example is an analyte that has a catalytic function that has the potential of altering the concentration of an effector by acting on a reporter substate. Catalytic analytes are often enzymes.

The term “target biomolecule” refers to a substance to be detected in a biological sample, or a sample potentially containing biological material. The target biomolecule is an analyte that is part of a sample.

The term “reporter” or “reporter molecule” will mean a substance capable of reacting with a substrate to alter the concentration of an effector molecule in an environment. The reporter may be chemically or catalytically based. Typical reporter molecules of the invention are enzymes.

The term “reporter substrate” refers to a substrate of the reporter molecule (e.g. enzyme). The reporter substrate is involved in the reporter function which results in the effector molecule being produced or consumed. Typical reporter substrates are enzyme substrates.

The term “effector” or “effector molecule” refers to a small molecule that has the ability to alter the conductance of a semiconducting CNT. Thus changes in the concentration of the effector can be detected by monitoring changes in the conductance of the CNT.

The term “support” refers to any material comprised within the nanosensor that will serve as a support for the various elements of the sensor, such as the CNTs. The term “surface” refers to the outer portion of a support or the carbon nanotube.

The term “source electode” will mean one of the three terminals of a field effect transistor from which the majority carrier flows into the transistor.

The term “drain electode” will mean one of the three terminals of a field effect transistor through which the majority carrier exits the transistor.

The term “gate electode” will mean one of the three terminals of a field effect transistor which by means of an electric field controls the flow of charge carriers in the transistor, thereby controlling the output current.

The term “polypeptide” refers to a chain of amino acids which may be an entire protein or may be a portion thereof. Polypeptides may be natural or synthetic, and may include one or more artificial chemical analogues of a naturally occurring amino acid. For the purposes of this description, a peptide is considered to be a type of polypeptide and a polypeptide is a type of protein.

A “oligonucleotide” or “oligo” is a polymer of RNA, DNA, or peptide nucleic acid (PNA). It optionally contains synthetic, non-natural or altered nucleotide bases. The base sequence of an oligonucleotide probe is complementary to the sequence of the portion of the target nucleic acid molecule to which hybridization is desired. An oligonucleotide probe may also be used to bind to a nucleic acid binding protein. In this case it may be double-stranded if interaction with the binding protein requires a double-strand structure. An oligonucleotide may also be covalently linked to a protein.

The term “peptide nucleic acid” refers to a material having nucleotides coupled together by peptide linkers.

A nucleic acid molecule is “hybridizable” to another nucleic acid molecule, such as a cDNA, genomic DNA, or RNA, when a single stranded form of the nucleic acid molecule can anneal to the other nucleic acid molecule under the appropriate conditions of temperature and solution ionic strength. Hybridization and washing conditions are well known and exemplified in Sambrook, J., Fritsch, E. F. and Maniatis, T. Molecular Cloning: A Laboratory Manual, Second Edition, Cold Spring Harbor Laboratory Press, Cold Spring Harbor (1989), particularly Chapter 11 and Table 11.1 therein. The conditions of temperature and ionic strength determine the “stringency” of the hybridization. Stringency conditions can be adjusted to screen for moderately similar fragments, such as homologous sequences from distantly related organisms, to highly similar fragments, such as genes that duplicate functional enzymes from closely related organisms. Post-hybridization washes determine stringency conditions. One set of preferred conditions uses a series of washes starting with 6×SSC, 0.5% SDS at room temperature for 15 min, then repeated with 2×SSC, 0.5% SDS at 45° C. for 30 min, and then repeated twice with 0.2×SSC, 0.5% SDS at 50° C. for 30 min. A more preferred set of stringent conditions uses higher temperatures in which the washes are identical to those above, except for the temperature of the final two 30 min washes in 0.2×SSC, 0.5% SDS was increased to 60° C. Another preferred set of highly stringent conditions uses two final washes in 0.1×SSC, 0.1% SDS at 65° C. Hybridization requires that the two nucleic acids contain complementary sequences, although depending on the stringency of the hybridization, mismatches between bases are possible. The appropriate stringency for hybridizing nucleic acids depends on the length of the nucleic acids and the degree of complementation, variables well known in the art. The greater the degree of similarity or homology between two nucleotide sequences, the greater is the value of Tm for hybrids of nucleic acids having those sequences. The relative stability (corresponding to higher Tm) of nucleic acid hybridizations decreases in the following order: RNA:RNA, DNA:RNA, DNA:DNA. For hybrids of greater than 100 nucleotides in length, equations for calculating Tm have been derived (see Sambrook et al., supra, 9.50-9.51). For hybridizations with shorter nucleic acids, i.e., oligonucleotides, the position of mismatches becomes more important, and the length of the oligonucleotide determines its specificity (see Sambrook et al., supra, 11.7-11.8).

Nanosensors

The nanosensors of the invention involve a homogeneous reporting system for the detection of an analyte. The main elements of the nanosensor of the invention are:

An electrically conducting path between at least two electrodes comprised of at least one semiconducting CNT where the CNT has a baseline conductance;

An effector molecule in association with the CNT where alterations in the concentration of the effector will alter the conductance of the CNT; and

Optionally, a reporter, typically a catalyst, that changes the concentration of the effector. An analyte may itself act as the reporter.

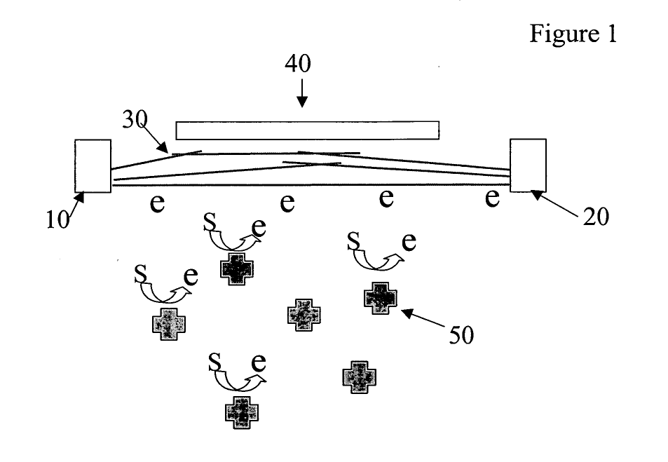

The invention may best be understood by making reference to the diagrams. For example, one embodiment is shown in FIG. 1. As shown in FIG. 1 the nanosensor comprises two electrodes (10, 20)connected by an electrically conducting path comprising at least one semiconducting CNT (30). The CNT inherently possesses a baseline conductance. The electrodes (10, 20) may be independently either source or drain. The CNT (30) is in association with an effector molecule (e). The nanosensor additionally may comprise a gate electrode (40) which generates an electric field to gate the conductance of the CNTs. An analyte is introduced to the nanosensor which is a substrate (S) of a reporter molecule (50). The substrate (S), which is also the analyte, is acted on by the reporter (50) whereby the concentration of the effector (e) is altered, producing a corresponding alteration in conductance of the CNT (30). Changes in the conductance of the CNT (30) indicate the presence and quantity of the analyte.

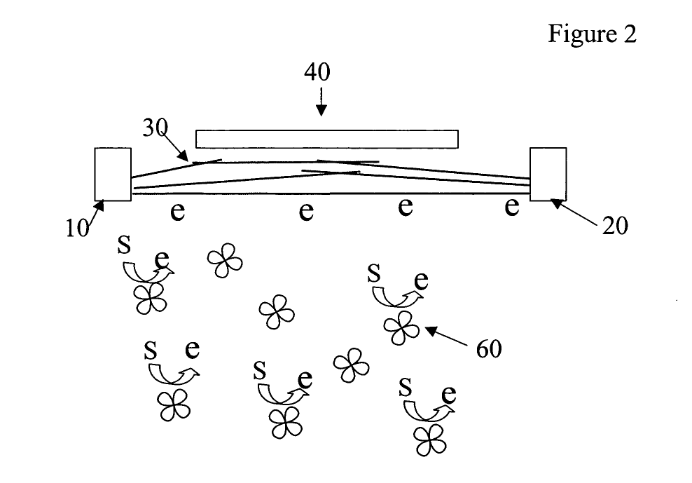

Another embodiment applicable to the detection of a catalytic analyte is shown in FIG. 2. The basic elements of the nanosensor are as illustrated in FIG. 1. A catalytic analyte (60) functions as a reporter and acts on an added reporter substrate (S) that is designed to interact with the catalytic analyte, thereby causing a change in concentration of an effector (e). The concentration of the effector (e) is altered, producing a corresponding alteration in conductance of the CNT (30). Changes in the conductance of the CNT (30) indicate the presence and quantity of the analyte.

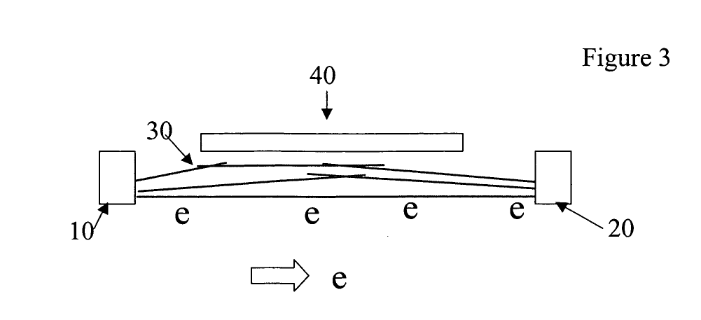

Another embodiment applicable to the direct detection of an analyte is shown in FIG. 3. The basic elements of the nanosensor are as illustrated in FIG. 1. An analyte is introduced that is itself an effector (e) and thus its addition directly changes the concentration of the effector (e). The CNT is in contact with the effector, such that its conductance is altered due to the change in the concentration of the effector thereby detecting the presence of the analyte.

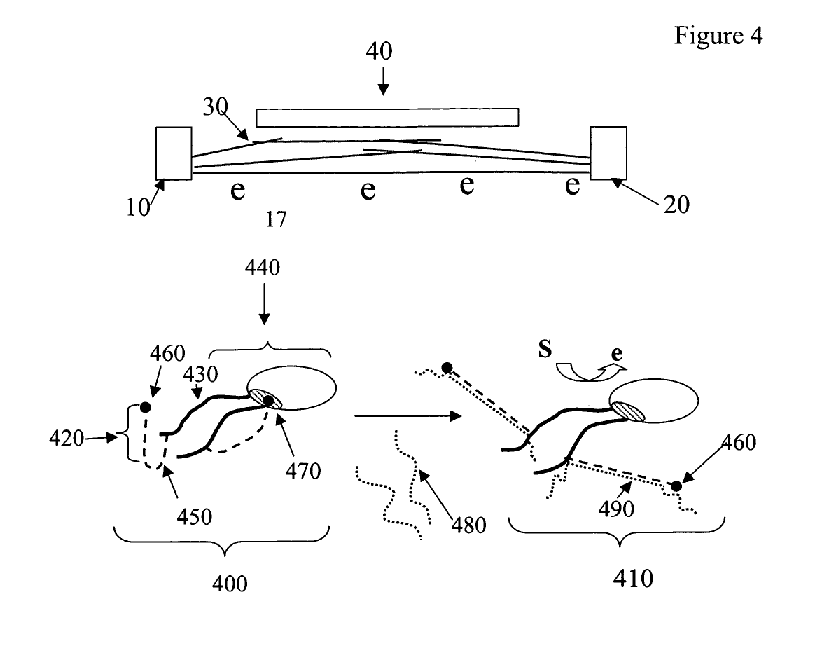

In those instances where the reporter is catalytic, the invention provides a format for the nanosensor where the reporter may be activated and “switched on” by the presence of an analyte. Analytes suitable for detection via an activity switch reporter will be those that have the ability to interact with the reporter and “switch on” the reporter. This format employs what is referred to herein as an “activity switch” and allows greater flexibility in the design of the sensor. A specific embodiment of the activity switch is illustrated in FIG. 4. The basic elements of the nanosensor are as illustrated in FIG. 1. In this embodiment the reporter is an enzymatic glycoprotein (440), and may exist in either an active (410) or inactive (400) form. One aspect of the glycoprotein (440), is the presence of a point of attachment for an activity switch (420), such as an oligosaccharide chain (430). The activity switch comprises an oligonucleotide (450) which is an analyte receptor anchored via its 5′ end to the glycoprotein (440), and an inhibitor (460) attached to the oligo at the 3′ end. The oligonucleotide is highly flexible and in its single stranded form is able to bend such that the inhibitor binds to the active site of the protein (470) resulting in the inactive form (400) of the enzymatic glycoprotein (440). When the inhibited reporter comes in contact with a nucleic acid analyte (480) that is complementary to a portion of the anchored oligo analyte receptor (450) the resulting hybridization (490) pulls the inhibitor (460) away from the active site of the glycoprotein (440), thus switching on the enzyme which then acts on the substrate to change the concentration of the effector.

In each of these embodiments no immobilization of the analyte is necessary for detection, greatly enhancing the utility of the assay over those methods performed in heterogeneous format for some applications.

Carbon Nanotubes of the Nanosensor

The nanosensor of the invention comprises at least one semiconducting CNT comprised within an electrically conducting path. CNTs have diameters on the nanometer scale and a ratio of the length to the diameter, i.e., the aspect ratio, of at least 5. In general, the aspect ratio is between 100 and 100,000. Carbon nanotubes are single-walled hollow cylinders composed primarily of carbon atoms. CNTs of the nanosensors of the invention may be doped with agents such as metals and may have coatings. Preferred CNTs are free of metals.

CNTs may be produced by a variety of methods known to those skilled in the art, and are additionally commercially available. Methods of CNT synthesis include laser vaporization of graphite (A. Thess et al. (1996) Science 273:483), arc discharge (C. Journet et al. (1997) Nature 388:756) and HiPCo (highpressure carbon monoxide) process (P. Nikolaev et al. (1999) Chem. Phys. Lett. 313:91). Chemical vapor deposition (CVD) can also be used for producing carbon nanotubes (J. Kong et al. (1998) Chem. Phys. Left. 292:567; J. Kong et al. (1998) Nature 395:878; A. Cassell et al. (1999) J. Phys. Chem. 103, 6484-6492; H. Dai et al. (1999) J. Phys. Chem. 103:11246).

Additionally CNTs may be grown via catalytic processes both in solution and on solid substrates (Yan Li, et al. (2001) Chem. Mater. 13(3):1008; N. Franklin and H. Dai (2000) Adv. Mater. 12:890; A. Cassell et al. (1999) J. Am. Chem. Soc. 121:7975).

Preferred in the invention are single-walled CNTs. The CNTs are placed in a conducting path between two electrodes, generally the source and drain. A variety of types of CNTs may be used where at least one of the CNTs between source and drain electrodes is semiconducting to provide an electrically conducting path that can be controlled by a gating electrode. Multiple CNTs of varying chirality may be joined to provide the electrically conducting path.

The CNTs may be suspended between the source and drain electrodes of the nanosensor or supported on a suitable support surface. The support surface may be comprised of any non-conductive material. Supports are common and well know in the art and will include, but are not limited to materials such as silicon, silicon dioxide, silicon nitride,polysilicon, polymeric materials, glass, agarose, nitrocellulose, nylon, ferromagnetic materials, carbon, metals and insulating materials as well as semiconducting materials. Particularly useful are silica chips. Typically silica chips have a thin layer of natural oxide, which has very low electrical conductivity and is an insulator. For better insulation of the surface from the underlying silica, a thicker oxide layer that is typically about 500-600 nm may be added, by a method such as with a thermal treatment in air. This provides additional insulation from the underlying silica.

A gating electrode in the nanosensor generates an electric field to change the CNT conductance such that its sensitivity to the presence of the effector can be optimized. The gate is an electrode separated from the CNT by a dielectric material and polarized relative to the drain electrode. The gate may be for example a back gate, top gate or split gate for operation in air. Alternatively, an electrode that contacts a solution in the CNT chamber may be used for operation as a liquid gate.

Since the concentration of an effector in solution provides the signal for detection by the CNT, there is no requirement for close proximity between the CNT and the analyte. This feature allows the CNT to be in any location accessible either by diffusion or flow, including such as by pumping and injecting, of the effector in solution. For example, the CNT may be in the same chamber where the effector concentration is changed, or in a separate chamber.

The surface of the CNT may be functionalized or coated to enhance or increase the specificity of the detection of the effector small molecule. Coatings such as PEG, PEI, PFE, polylysine, polyglutamic acid, and polystyrene sulfonic acid may be added to control non-specific binding or the binding of charged species.

The exact structure of the nanosensor is not specified by the nanosensor of the invention. Any sensor structure may be employed with the components of the invention wherein the CNT comes in contact with the solution in which the effector concentration is changed.

Analytes

Analytes that are targets may be, for example, chemicals and biomolecules. Biomolecules are particularly suitable analyte targets of the invention. Any biomolecule which can change the concentration of an effector either directly, or in conjunction with a reporter substrate or a reporter molecule, is an analyte for the purposes of the invention. Additionally any analyte that can interact with the analyte receptor in an activity switch such that the reporter molecule modified with the activity switch is activated is an analyte for the purposes of the invention. A target biomolecule may for example be an enzyme that catalyzes a reaction involving an effector, a metabolite that reacts with an effector in the presence of an enzyme, a metabolite that reacts with an effector, and a nucleic acid that can bind the analyte receptor in an activity switch such that the reporter molecule modified with the activity switch is activated. If the analyte is a double stranded nucleic acid, prior to detection, the double stranded DNA is melted into two free single strands. Binding of a nucleic acid single strand and the steps that follow are carried out below the melting temperature.

Reporter Molecule and Reporter Substrate

A reporter molecule is a part of the nanosensor as shown in FIG. 1, where the analyte is a substrate of the reporter molecule. A reporter substrate is a part of the nanosensor as shown in FIG. 2, where the analyte is a catalyst. The nanosensor in FIG. 4 incorporates both a reporter substrate and a reporter molecule that is modified, as described below, with an activity switch.

The reporter molecule may be any molecule that alters the concentration of an effector in solution in the presence of an analyte. The effector is either produced to increase its concentration, or consumed to decrease its concentration as a result of that interaction. Reporter molecules may be enzymes having an analyte as a substrate. The enzyme reporter molecule catalyzes a reaction involving the analyte that results in a change in the concentration of the effector.

Oxidases such as glucose oxidase, laccase, bilirubin oxidase, alphahydroxy acid oxidase, aldehyde oxidase, L-amino acid oxidase, ascorbate oxidase, cholesterol oxidase, and xanthine oxidase can be used as reporter molecules with analytes that are oxidizable. For example, glucose oxidase catalyzes the oxidation of glucose to produce hydrogen peroxide and gluconolactone. This reaction decreases the concentration of the effector molecule, oxygen, which is detected by the CNT. Laccase reduces oxygen to water in the presence of oxidizable analyte substrates such as ascorbate, phenols and quinols, thereby decreasing the concentration of oxygen in solution. Ammonia production is accomplished for example using as the reporter molecule/analyte combination: glutaminase/glutamine, asparaginase/asparagine, and urease/urea. Other examples of ammonia producing reporter molecules are amidase, formamidase, arginase, and ammonia lyases. Decreases in concentration of the effector molecule ammonia can be accomplished using for example reporter molecules glutamine synthase and asparagine synthase with glutamatic acid and aspartic acid as analytes, respectively.

The effector molecule nitrogen dioxide may be produced using nitric oxide synthase; the nitric oxide produced will be converted to nitrogen dioxide in the presence of oxygen. The concentration of the effector H⁺ may be changed using as reporter molecules enzymes such as urease and various types of esterases, nucleases, and phosphatases which act on their analyte substrates and cause the H⁺ concentration to be decreased (pH to increase).

More than one reporter molecule may be used in a cascade of reactions that alter the concentration of an effector in the presence of an analyte. Examples are the combination of glucose oxidase and catalase that result in the oxidation of glucose to gluconolactone and the consumption of O₂ but without accumulation of H₂O₂. Similarly D-amino acid oxidase and monoamine oxidases each produce H₂O₂ and NH₃. The presence of catalase would assure the disproportionation of H₂O₂ to O₂ and water, resulting in a both consumption of O₂ and production of NH₃. These two effects reinforce each other to switch on the carbon nanotube conductance at more positive gate voltages.

Reporter substrates may be enzyme substrates, where the analyte is an enzyme that catalyzes a reaction involving the reporter substrate that results in a change in the concentration of the effector. The examples of enzymes and substrates given above as reporter molecules and analytes may be used where the analyte is the enzyme and the substrate is the reporter substrate. For example, oxidizable reporter substrates are used with analytes that are oxidases.

Activity Switch

The reporter molecule may be modified to include an activity switch that can regulate the enzymatic activity of the reporter. The activity switch has two components: an inhibitor that binds to the active site or to an allosteric site of the reporter enzyme thereby blocking its activity, and an analyte receptor that binds to the target analyte. An enzyme having an activity switch is an activity switch derivatized enzyme. The activity switch may be attached to the reporter molecule directly to the protein. Direct attachment may be, for example, through a lysine using an amine group, through a cysteine using a thiol group, through as aspartic acid or a glutamic acid using a carboxyl group by methods known to one skilled in the art. If the reporter molecule has oligosaccharide chains (as in a glycoprotein), the activity switch may be attached to these chains. For example, the enzymatic glycoproteins glucose oxidase and laccase have oligosaccharide chains which are locations for activity switch attachment.

In the present invention, the analyte receptor may be any molecule which can bind to the target analyte and which allows the inhibitor to access the active site or allosteric site in the free state but does not allow access upon binding to the target analyte. The analyte receptor may be, for example, a protein, a polypeptide an oligopeptide, a peptide nucleic acid, an oligonucleotide, a polynucleotide or any type of nucleic acid. Preferred is a single stranded oligonucleotide probe, attached via the 5′ end to the reporter molecule and linked at the 3′ end to an inhibitor of the enzyme activity. It is understood that the attachments at the 5′ and 3′ ends can be switched without impact on the function. Any methods for attaching compounds to DNA, and DNA to proteins may be used to prepare an enzyme switch. The oligonucleotide, which is highly flexible in its single stranded form, is able to bend such that the inhibitor binds to the active site or to the allosteric site, blocking the action of the enzyme on its reporter substrate. Upon hybridization of the complementary strand of the analyte DNA (or RNA) to the enzyme-bound oligonucleotide probe, the double stranded DNA (or DNA/RNA hybrid) is now much more rigid than the single strand, with a persistence length some 60-fold greater than that of the single stranded probe oligonucleotide. The inhibitor can then no longer bind to the active site of the enzyme, which is turned on (see diagram in FIG. 4). The active enzyme is now able to process a reporter substrate and change the concentration of the effector. Combinations of enzymes and reporter substrates described above as reporter molecule/analyte combinations may be used. One skilled in the art will know the length of analyte receptor required to have stable hybridization and the conditions of the assay required to maintain the double strand during detection. It is particularly useful for hybridization of the oligonucleotide analyte receptor to the analyte nucleic acid to drive the dissociation of the inhibitor from its binding site. This occurs when the decrease in free energy associated with hybridization of the analyte receptor to the analyte exceeds that associated with the binding of the inhibitor to the enzyme.

Direct Analyte Detection

An analyte may directly change the concentration of the effector without adding a reporter molecule or reporter substrate. For example, aldehydes, ketones, alkynes and acid chlorides react with ammonia. These types of analytes would themselves reduce the concentration of ammonia when added to a solution containing ammonia. A solution may be pre-loaded with ammonia in order to detect the presence of this type of analyte. As another example, dienes undergo an autooxidative and photooxidative reaction in the presence of oxygen. Thus a diene analyte incubated in a solution containing oxygen would reduce the concentration of the effector molecule oxygen under illumination.

Effector

Small molecules that have the ability to change the conductance of a semiconducting CNT may be used as effectors in this invention. It is known that oxygen (O₂) and ammonia (NH₃) each are able to significantly change the conductance of CNTs. In addition nitrogen dioxide (NO₂), which is spontaneously formed from nitric oxide and oxygen, can change the conductance of CNTs allowing this small molecule to be an additional effector. Also Hydrogen ions (H+) may be used as an effector, since a change in their concentration affects the conductance of CNTs. Other small molecules may be identified as being able to change the properties of CNTs, and as such may also be appropriate effectors for use in this invention. The effector is in solution that must be in contact with the CNT. Typically effectors are consumed or produced by the interaction of a reporter and a reporter substrate.

Samples

Samples that may be assayed for the presence of an analyte using nanosensors and methods of the present invention include biological samples as well as non-biological samples. For example, a sample may be from a cell, tissue or fluid from a biological source including a human, an animal, a plant, fungus, bacteria, virus, etc. The source of a sample is not limited and may be from an environmental source, from food or feed, produced in a laboratory, or other source.

Method for Analyte Detection

In the method for analyte detection, a sample is placed in contact with a reporter molecule or a reporter substrate and the effector concentration is changed as a result. Alternatively, the concentration of the effector is changed by the analyte alone. In still another alternative, the analyte is placed in contact with a reporter substrate and a reporter molecule that is modified with an activity switch such that the reporter is initially inactive, and it is activated in the presence of the analyte whereby the effector concentration is changed. The solution with the altered effector concentration may already be in contact with the CNT or the solution with the altered effector concentration is brought in contact with the CNT. The solution containing the effector may flow through a channel, tubing, or other conduit to come in contact with the CNT.

The conductance of the CNT is measured and compared to a measure of the CNT conductance that was taken prior to adding the sample or at the earliest time following the addition of the sample (baseline conductance). Measurement of the CNT conductance is generally made by applying a dc (direct current) bias voltage between the source and drain electrodes while varying the gate voltage. In addition, the signal to noise ratio may be improved by ac (alternating current) modulation of the bias voltage. Alternatively, the CNT conductance is measured by holding the gate voltage constant and recording the current as a function of time. A gate electrode is preferred but not required.

### EXAMPLES

The present invention is further defined in the following examples. It should be understood that these examples, while indicating preferred embodiments of the invention, are given by way of illustration only. From the above discussion and these examples, one skilled in the art can ascertain the essential characteristics of this invention, and without departing from the spirit and scope thereof, can make various changes and modifications of the invention to adapt it to various uses and conditions.

The meaning of abbreviations used is as follows: “min” means minute(s), “μL” means microliter(s), “mL” means milliliter(s), “nm” means nanometer(s), “mm” means millimeter(s), “cm” means centimeter(s), “μm” means micrometer(s), “mM” means millimolar, “M” means molar, “V” means volts, “mV” means millivolts, “Vg” means gate voltage, “Vsd” means source-drain voltage, “Isd” means source-drain current, “p-type” means charge carrier type (e.g. hole), “CVD” means chemical vapor deposition.

### Example 1

### Carbon Nanotube Response to Oxygen in Gas Phase

Nanotube devices, prepared as follows, were purchased from Molecular Nanosystems (Palo Alto, Calif.). Single-walled carbon nanotubes were grown from catalyst pads in a CVD furnace at 900° C. The catalyst pads were patterned on a thermally oxidized surface (500 nm thick) of a (100) silicon wafer. After the growth, less than or equal to 5 nm of Ti, 50 nm of Pd and less than 50 nm of Au layers were deposited sequentially onto the SiO₂/Si surface to form electrical contacts with the carbon nanotubes.

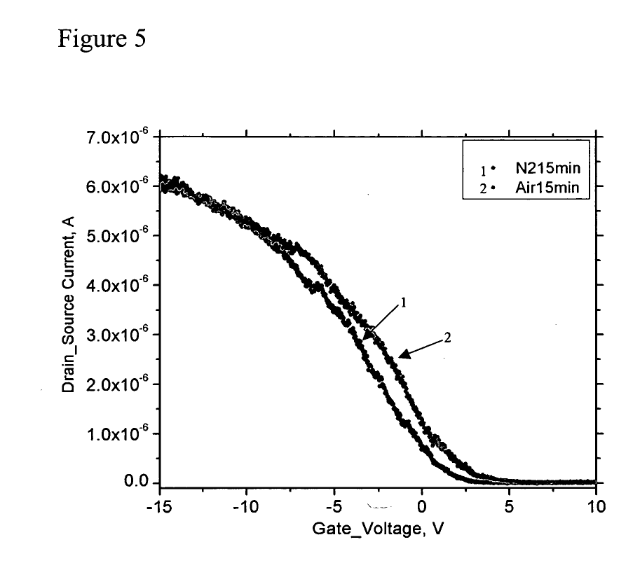

The metallic nanotubes present in the gap (2 micron) were destroyed, by ramping the bias voltage from 0 to 10V while holding the back gate voltage at 0V. This procedure performed in air, enhanced the ON-OFF ratio of the devices to ˜3-4 orders of magnitude. The electronic properties of the remaining semiconducting nanotubes were monitored by applying a dc bias voltage between the source and the drain electrodes while changing the back gate voltage. A flow cell of 4.4 μl volume was mounted and sealed around the cabon nanotube device using an O-ring to allow control of the surrounding atmosphere. The nanotube devices were first characterized in air and then under nitrogen atmosphere. The plots of the source-drain current vs gate voltage recorded in air and nitrogen are presented in FIG. 5. The plot recorded in nitrogen atmosphere was shifted toward negative gate voltages relative to the plot recorded in air. This shift is explained by the following events. As nitrogen gas is passing through the flow cell placed on top of the nanotube device, the amount of oxygen in the atmosphere and that attached to the nanotube decreases with time. The removal of oxygen from the sidewalls of the nanotube results in an injection of electrons back into the nanotube, where electron-hole recombination takes place spontaneously thus decreasing the concentration of the free p-type carriers, which makes the device harder to turn ON. The turn ON voltage depends greatly on the environment of the nanotube and also reflects the concentration of the free charge carriers in the nanotube. As soon as the nitrogen gas is replaced with air, the current vs gate voltage plot shifts back to the original position recorded previously indicating that oxygen was reunited with the nanotube.

This experiment, carried out in gas phase, clearly indicated that carbon nanotube based devices are adequately sensitive to oxygen and can be used for oxygen-mediated sensing applications.

### Example 2

### Carbon Nanotube Response to Oxygen in Liquid Environment

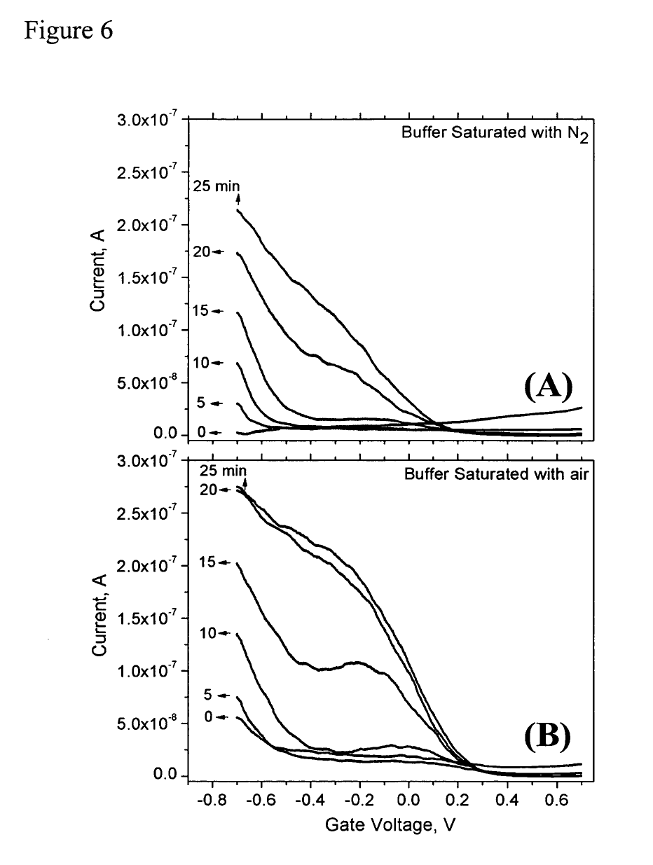

In the previous example, the source-drain current was monitored as a function of the gate voltage applied to the back gate. Here liquid gating was used to control the conductance of the nanotube. To operate in liquid gating mode a third electrode, in addition to the source and the drain electrodes, was submerged in solution that was injected into the flow cell chamber. The source-drain current vs liquid gate voltage characteristic in 50 mM glycine buffer at pH=3 was similar to the characteristic recorded in air using the back gate. However, once the solution of a 1 to 1 ratio of the redox couple ferricyanide (K₃Fe(CN)₆)/ferrocyanide (K₄Fe(CN)₆) (1 mM total concentration) was added into the cell chamber, a large shift toward more negative gate voltages occurred immediately. This shift is due to the reduction of the carbon nanotube at the redox potential defined by the ferricyanide/ferrocyanide ratio. After equilibration, the cell chamber was washed thoroughly with fresh 50 mM glycine buffer to remove ferro- and ferricyanide molecules. Recovery was monitored over time in the presence and absence of oxygen. The rate of recovery depended greatly on the concentration of oxygen in the buffer solution. FIG. 6 shows the source-drain current as a function of liquid gate voltages recorded 5, 10, 15, 20 and 25 minutes after fresh buffer that had been equilibrated with nitrogen (FIG. 6A) or with air (FIG. 6B) was injected into the cell chamber. In the case of air-equilibrated buffer, the recovery was rapid and the lsd vs gate plot shifted halfway back after 15 minutes. However, in the case of nitrogen-equilibrated buffer the recovery was slowed and the Isd vs gate plot shifted halfway after 20 minutes. The total recovery was also greater in the buffer equilibrated with air than with that equilibrated with nitrogen. The faster recovery can be explained by the oxidation of the nanotube by oxygen molecules present in the air-equilibrated buffer. The oxidation of the carbon nanotube causes an increase in the number of p-type charge carriers and makes the nanotube more p-type, thus producing a shift toward positive gate voltages.

In this example the sensitivity of carbon nanotubes to oxygen molecules in a liquid phase environment was demonstrated. Based on this finding, biomolecules such as DNA can be detected by their induced attachment of a reporter molecule that consumes oxygen.

### Example 3

### Carbon Nanotube Response to Hydrogen Ions in Liquid Environment

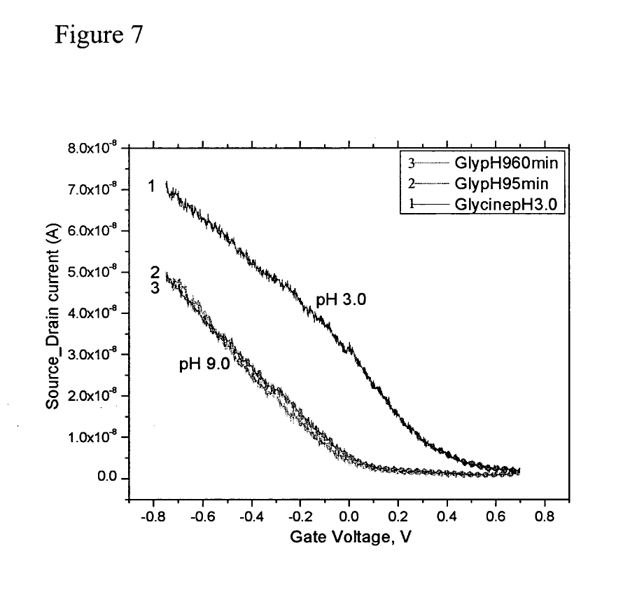

The liquid flow cell mounted on the nanotube device was initially filled with a 50 mM glycine buffer pH 3.0 and the source-drain current vs liquid gate voltage characteristic was recorded. The Isd vs. Vg plot is shown in FIG. 7 as curve 1. The buffer was then replaced with 50 mM glycine buffer, pH 9.0 over a period of 5 min and the Isd vs.Vg curve was re-measured under the same conditions (FIG. 7, curve 2). A shift of the Isd vs Vg curve toward negative gate voltages occurred. An additional incubation for 55 min under the same conditions produced little further change in the lsd vs.Vg curve (FIG. 7, curve 3).

The response of the nanotube to the pH of the solution makes it possible to use the nanotube as a pH sensor and a detector for any process that results in a change in pH, or hydrogen ion concentration.

1. A nanosensor for detecting the presence of an analyte comprising:
a) at least two electrodes connected by an electrically conducting path comprised of one or more carbon nanotubes wherein at least one of said carbon nanotubes is semiconducting, and wherein the carbon nanotube is in contact with an effector; and
b) at least one reporter molecule having an analyte as a reporter substrate.

2. A nanosensor for detecting the presence of a catalytic analyte comprising:
a) at least two electrodes connected by an electrically conducting path comprised of one or more carbon nanotubes wherein at least one of said carbon nanotubes is semiconducting, and wherein the carbon nanotube is in contact with an effector; and
b) a reporter substrate that is a substrate of an catalytic analyte.

3. A nanosensor for detecting the presence of an analyte comprising:
a) at least two electrodes connected by an electrically conducting path comprised of one or more carbon nanotubes wherein at least one of said carbon nanotubes is semiconducting, wherein the carbon nanotube is in contact with an effector; and
b) an effector responsive to the presence of an analyte.

4. A nanosensor for detecting the presence of an analyte comprising:
a) at least two electrodes connected by an electrically conducting path comprised of one or more carbon nanotubes wherein at least one of said carbon nanotubes is semiconducting, wherein the carbon nanotube is in contact with an effector;
b) a reporter molecule comprising an activity switch comprising an analyte receptor linked to a reporter inhibitor; and
c) a reporter substrate that is a substrate of the reporter molecule.

5. A nanosensor according to any of claims 1, 2, 3, or 4 optionally comprising a gate electrode.

6. A nanosensor according to any of claims 1, 2, 3, or 4 wherein the carbon nanotube is suspended between at least two electrodes.

7. A nanosensor according to any of claims 1, 2, 3, or 4 wherein the carbon nanotube is supported on a support.

8. A nanosensor according to claim 7 wherein the support is comprised of materials selected from the group consisting of silicon, polysilicon, silicon dioxide, silicon nitride, polymeric materials, glass, agarose, nitrocellulose, nylon, insulating materials.

9. A nanosensor according to claim 1 or 4 wherein the reporter molecule is an enzyme.

10. A nanosensor according to claim 2 wherein the analyte is an enzyme.

11. A nanosensor according to either of claims 9 or 10 wherein the enzyme is selected from the group consisting of glucose oxidase, laccase, ascorbate oxidase, bilirubin oxidase, glutaminase, alphahydroxy acid oxidase, aldehyde oxidase, L-amino acid oxidase, ascorbate oxidase, cholesterol oxidase, and xanthine oxidase and asparaginase.

12. A nanosensor according to any of claims 1, 2, 3, or 4 wherein the effector is selected from the group consisting of oxygen, ammonia, nitrogen dioxide, and hydrogen ions.

13. A nanosensor according to claim 2 or 4 wherein the reporter substrate is selected from the group consisting of glucose, bilirubin, ascorbate, glutamine, and asparagine.

14. A nanosensor according to any of claims 1, 2, 3, or 4 wherein the carbon nanotube is substantially free of metal.

15. A method for detecting an analyte comprising:
a) providing a nanosensor comprising:
i) at least two electrodes connected by an electrically conducting path comprised of one or more carbon nanotubes wherein at least one of said carbon nanotubes is semiconducting, and wherein the carbon nanotube is in contact with an effector and has a baseline conductance; and
ii) a reporter molecule having an analyte as a substrate;
b) providing a sample suspected of containing an analyte;
c) contacting the sample of (b) with the reporter molecule of (a) wherein the concentration of the effector molecule is altered resulting in a change in the conductance of the carbon nanotube with respect to the baseline conductance; and
d) measuring the change in conductance of the carbon nanotube with respect to the baseline conductance whereby the presence of the analyte is detected.

16. A method for detecting a catalytic analyte comprising:
a) providing a nanosensor comprising:
i) at least two electrodes connected by an electrically conducting path comprised of one or more carbon nanotubes wherein at least one of said carbon nanotubes is semiconducting, and wherein the carbon nanotube is in contact with an effector and has a baseline conductance; and
ii) a reporter substrate that is a substrate of a catalytic analyte;
b) providing a sample suspected of containing a catalytic analyte;
c) contacting the sample of (b) with the reporter substrate of (a) wherein the concentration of the effector molecule is altered resulting in a change in the conductance of the carbon nanotube with respect to the baseline conductance; and
d) measuring the change in conductance of the carbon nanotube with respect to the baseline conductance whereby the presence of the catalytic analyte is detected.

17. A method for detecting an analyte comprising:
a) providing a nanosensor comprising:
i) at least two electrodes connected by an electrically conducting path comprised of one or more carbon nanotubes wherein at least one of said carbon nanotubes is semiconducting, and wherein the carbon nanotube is in contact with an effector and has a baseline conductance; and
ii) an effector responsive to the presence of an analyte;
b) providing a sample suspected of containing an analyte;
c) contacting the sample of (b) with the effector of (a) wherein the concentration of the effector molecule is altered resulting in a change in the conductance of the carbon nanotube with respect to the baseline conductance; and
d) measuring the change in conductance of the carbon nanotube with respect to the baseline conductance whereby the presence of the analyte is detected.

18. A method for detecting an analyte comprising:
a) providing a nanosensor comprising:
i) at least two electrodes connected by an electrically conducting path comprised of one or more carbon nanotubes wherein at least one of said carbon nanotubes is semiconducting, and wherein the carbon nanotube is in contact with an effector and has a baseline conductance; and
ii) a reporter molecule having an activity switch comprising an analyte receptor linked to a reporter inhibitor;
b) providing a sample suspected of containing an analyte which binds to the analyte receptor of the activity switch wherein the reporter molecule becomes active;
c) contacting the sample of (b) with the reporter molecule of (a) wherein the concentration of the effector molecule is altered resulting in a change in the conductance of the carbon nanotube with respect to the baseline conductance; and
d) measuring the change in conductance of the carbon nanotube with respect to the baseline conductance whereby the presence of the analyte is detected.

19. A method according to any of claims 15, 16, 17, or 18 wherein the carbon nanotube is substantially free of metal.

20. A method according to any of claims 15, 16, 17, or 18 wherein the carbon nanotube is optionally supported on a surface.

21. A method according to claim 20 wherein the surface is comprised of materials selected from the group consisting of silicon, polysilicon, silicon dioxide, silicon nitride, polymeric materials, glass, agarose, nitrocellulose, nylon, insulating materials.

22. A method according to claim 15 or 18 wherein the reporter molecule is an enzyme.

23. A method according to claim 16 wherein the analyte is an enzyme.

24. A method according to either of claims 22 or 23 wherein the enzyme is selected from the group consisting of glucose oxidase, laccase, ascorbate oxidase, bilirubin oxidase, glutaminase, alphahydroxy acid oxidase, aldehyde oxidase, L-amino acid oxidase, ascorbate oxidase, cholesterol oxidase, and xanthine oxidase and asparaginase.

25. A method according to claim 16 or 17 or wherein the reporter substrate is selected from the group consisting of glucose, bilirubin, ascorbate, glutamine, and asparagine.
